(19) United States
(12) Patent Application Publication
(10) Pub. No.: US 2026/0272697 A1
(43) Pub. Date: Sep. 17, 2026

(54) CHEST AND ABDOMEN RESTRICTION DEVICE

(71) Applicant: UNIVERSITY OF FLORIDA RESEARCH FOUNDATION, INCORPORATED, Gainesville, FL (US)

(72) Inventors: Paul W. DAVENPORT, Gainesville, FL (US); Nicholas J. NAPOLI, Gainesville, FL (US); Victoria RIBEIRO RODRIGUES, Gainesville, FL (US)

(21) Appl. No.: 19/549,567

(22) Filed: Feb. 25, 2026

Related U.S. Application Data
(60) Provisional application No. 63/762,867, filed on Feb. 25, 2025.

(51) Int. Cl. A61F 5/37

(52) U.S. Cl. CPC A61F 5/3776

(57) ABSTRACT

An apparatus (100), including: a backrest (104); and at least one restriction assembly (200) having: a flexible strap (300) configured to wrap around a subject abutting a front of the backrest; a reel assembly (500) disposed behind the backrest and configured to tighten the flexible strap wrapped around the subject; and an anchor assembly (600) configured to hold the reel assembly apart from the backrest.

### STATEMENT REGARDING FEDERALLY SPONSORED DEVELOPMENT

This invention was made with government support under Grant No. N00014-22-1-2653 awarded by the Office of Naval Research. The government has certain rights in the invention.

### FIELD OF THE INVENTION

This invention relates to a restriction device suitable for restricting movement of a region of a torso.

### BACKGROUND OF THE INVENTION

Breathing is an essential activity performed by the human body, serving as the cornerstone for all other activities. When one is unable to breathe properly, it disrupts the balance between oxygen and carbon dioxide in the blood. In response to this, the human body initiates a cascade of effects, leading to a lack of attention, confusion, loss of consciousness, morbidity, and even mortality. The chest wall plays a crucial role in the breathing process, serving as the housing for the lungs and dynamically expanding and contracting to facilitate air exchange. Thus, any interference/restrictions with the chest wall disrupts the breathing mechanism. These restrictions may originate from physiological factors affecting thoracic compliance and muscle function or environmental factors, such as the impact of g-forces during high accelerations and water compression in deep diving etc. However, studying these conditions is challenging due to their complex nature.

Previous attempts to restrict the chest wall have been made. One of the earliest designs was a simple chest clamp used to limit the movement of the rib cage. The device consisted of two plywood boards, one placed on the posterior surface and the other on the anterior surface of the thorax [1]. This approach, however, fails to match the contour of the body and restricts the rib cage only along the sternum and the spine. A more effective alternative that addresses this limitation is chest wall strapping (CWS). This procedure involves restricting the thorax and abdomen using fabric, cinch straps, and/or a chest cast [2], [3], [4]. While CWS is commonly used today, it lacks the precision to quantify the load being added to the chest wall. Further, these designs are unable to measure how much the chest and/or the abdomen moves with a breath.

### SUMMARY OF THE INVENTION

In an example embodiment, the restriction device includes a backrest; and at least one restriction assembly including: a flexible strap configured to wrap around a subject abutting a front of the backrest; a reel assembly disposed behind the backrest and configured to tighten the flexible strap wrapped around the subject; and an anchor assembly configured to hold the reel assembly apart from the backrest.

The backrest may include at least one opening configured to permit the flexible strap to pass therethrough. The flexible strap may include a pressure sensor. The reel assembly may include a ratcheting reel assembly. The reel assembly may be configured to permit adjustment of a width of the flexible strap to conform to a width of the subject.

The anchor assembly may include a static load assembly configured to fix the reel assembly into a fixed position relative to the backrest. The restriction device may further include a guide assembly configured to enable movement of the reel assembly toward and away from the backrest. The static load assembly may be configured to lock the reel assembly into the fixed position along the guide assembly.

The anchor assembly may include a dynamic load assembly configured to apply a constant load to the reel assembly in a direction away from the backrest to hold the reel assembly apart from the backrest. The dynamic load assembly may include a constant force spring configured to apply the constant load.

The restriction device may include a displacement sensor configured to measure displacement of the reel assembly.

The at least one restriction assembly may include a chest restriction assembly configured to wrap around a chest of the subject; and an abdomen restriction assembly configured to wrap around an abdomen of the subject. The chest restriction assembly and the abdomen restriction assembly may be configured to operate independently of each other.

In an example embodiment, the restriction device includes a backrest; and at least one restriction assembly including: a flexible strap configured to form a closed loop around a subject abutting a front of the backrest; a guide assembly disposed behind the backrest; a reel assembly disposed on the guide assembly, configured to move toward and away from the backrest along the guide assembly, and configured to operate as a reel for the flexible strap; and an anchor assembly configured to hold the reel assembly apart from the backrest.

The flexible strap may include a pressure sensor.

The reel assembly may include a first attachment assembly configured to connect to the flexible strap. The first attachment assembly may include a ratchet assembly configured to reel the flexible strap in to apply tension to the flexible strap.

The reel assembly may include a first attachment assembly configured to connect to the flexible strap; a second attachment assembly configured to connect to the flexible strap; and a width adjustment assembly configured to enable adjustment of a width between the first attachment assembly and the second attachment assembly. The width adjustment assembly may include a width adjustment rail. The first attachment assembly and the second attachment assembly are slidably connected to the width adjustment rail in a manner effective to permit the adjustment of a distance therebetween to adjust the width.

The anchor assembly may include a static load assembly configured to fix the reel assembly into a fixed position relative to the guide assembly and thereby relative to the backrest.

The anchor assembly may include a dynamic load assembly configured to apply a constant load to the reel assembly in a direction away from the backrest and along the guide assembly to hold the reel assembly apart from the backrest.

The restriction device may include a displacement sensor configured to measure displacement of the reel assembly.

The at least one restriction assembly may include: a chest restriction assembly configured to wrap around a chest of the subject; and an abdomen restriction assembly configured to wrap around an abdomen of the subject. The chest restriction assembly and the abdomen restriction assembly may be configured to operate independently of each other.

### BRIEF DESCRIPTION OF DRAWINGS

FIG. 1 is an elevated perspective view of an example embodiment of a restriction device disclosed herein.

FIG. 2 is a top perspective view of the restriction device of FIG. 1.

FIG. 3 is a side view of the restriction device of FIG. 1.

FIG. 4 is a top view of the restriction device of FIG. 1.

FIG. 5 is a front view of the restriction device of FIG. 1.

FIG. 6 is a side view of a chest restriction assembly of the restriction device of FIG. 1.

FIG. 7 shows average reaction time across different loads.

FIG. 8 compares average peak airflow between baseline and static anchoring conditions.

FIG. 9 shows a decrease in average time of inspiration under static anchoring conditions versus baseline.

FIG. 10 shows the average peak inspiratory mouth pressure increase between baseline and static anchoring conditions

### DETAILED DESCRIPTION OF THE INVENTION

To address these limitations, the Inventors have developed a chest wall and abdomen restriction device designed to mimic static and dynamic chest and abdominal restrictions. The restriction device provides a controlled environment to study effects of chest and/or abdominal impairment on both breathing and cognition. The device features a strap that conforms to the body, ensuring an even load on the chest and abdomen areas. The device can precisely set a known load for the chest area and abdomen separately, eliminating the uncertainty associated with load application in CWS. Additionally, the device is equipped with a displacement sensor to measure the horizontal displacement of the chest wall and abdomen during breathing when aload is applied.

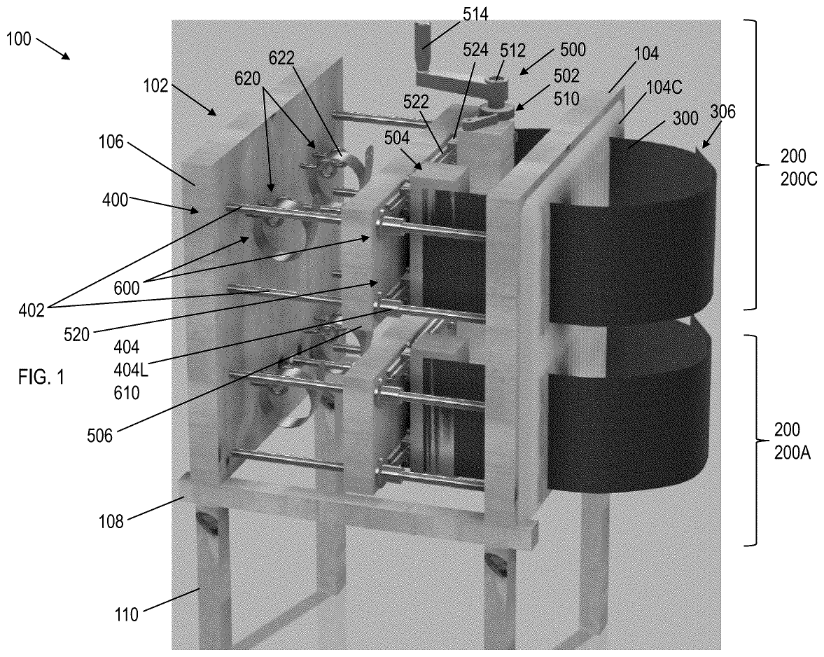

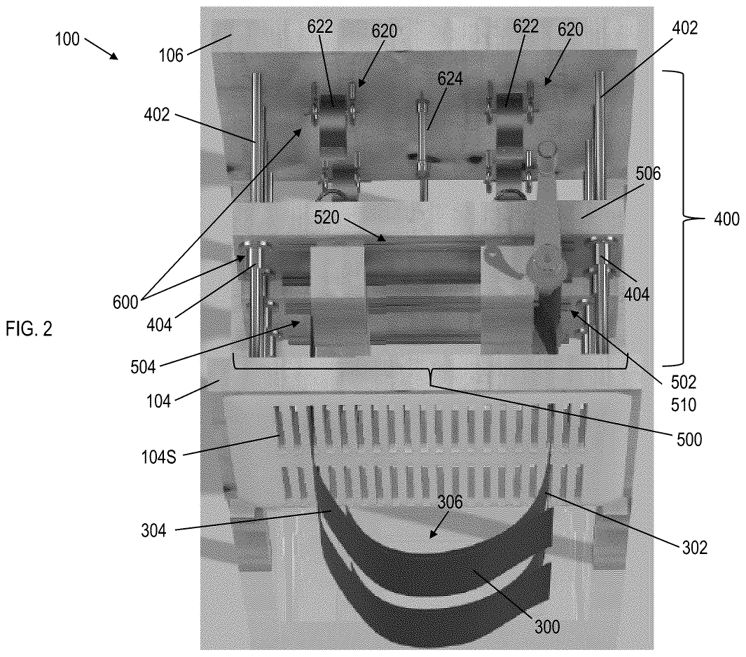

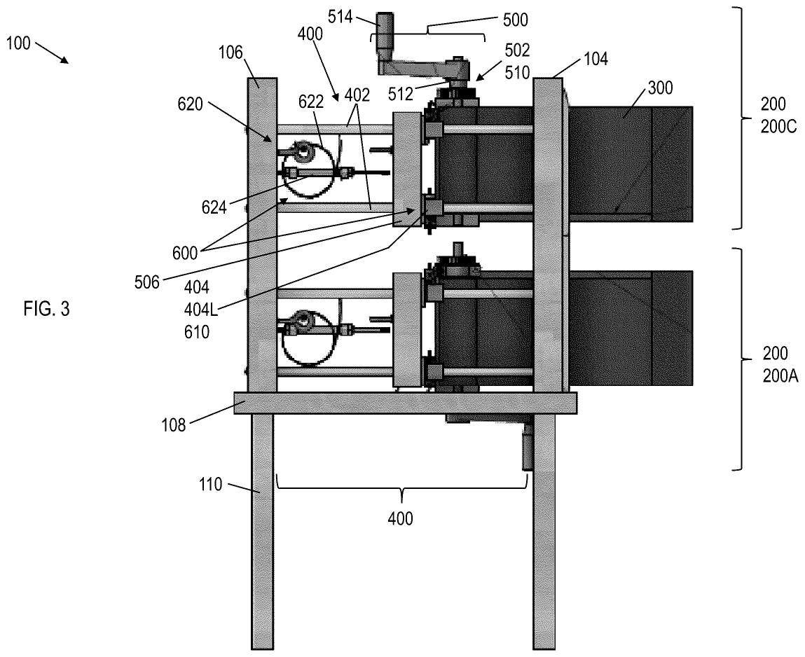

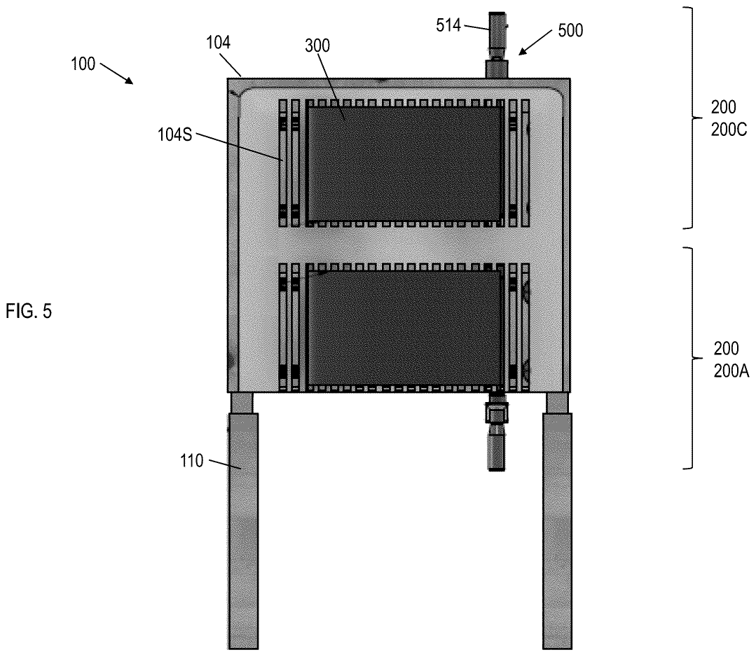

FIG. 1 to FIG. 5 show various views of an example embodiment of a restriction device disclosed herein.

The restriction device 100 includes a fixed framework 102 including a front plate 104 (e.g., a backrest) with optional cushioning 104C thereon, a rear plate 106, a base 108 that connects to the front plate 104 and to the rear plate 106, and legs 110. The restriction device 100 further includes at least one restriction assembly 200, such as one chest restriction assembly 200C, one abdomen restriction assembly 200A, or both a chest restriction assembly 200C and an abdomen restriction assembly 200A as is shown in FIG. 1.

In the example embodiment discussed below, the restriction assemblies 200C, 200A have the same components and function the same way. Parameters of the individual components may vary (e.g., belt length, belt width, spring constants etc.) to tailor the restriction assembly 200C, 200A to the respective portion of the torso being restricted. In another example embodiment, the restriction assemblies 200C, 200A are identical to each other. In yet another example embodiment, the restriction assemblies 200C, 200A may have different components but operate using the principles disclosed herein.

Each restriction assembly 200 includes a flexible strap 300; a guide assembly 400; a reel assembly 500; and an anchor assembly 600.

The flexible strap 300 is configured to conform to the contour of a human chest and/or a human abdomen 700. It may be composed of a flexible material such as fabric (e.g., a vinyl-like fabric). The flexible strap 300 may also be configured so that it does not stretch. A flexible strap 300 that does not stretch will transfer all movement of the chest/abdomen 700 and/or all tensile force in the flexible strap 300 to the reel assembly 500 without any loss due to stretching of the flexible strap 300.

The flexible strap 300 may have a first portion 302 and a second portion 304 that can be secured to each other via, for example, hook and loop fasteners or the like. In the example embodiment shown, a person sits with their back against the backrest 104. The first portion 302 and the second portion 304 of the flexible strap 300 are secured to each other to form a loop 306 that loops around the chest/abdomen 700. The flexible strap 300 may then be tightened around the chest/abdomen 700 as detailed below. A sensor 310 (e.g., a pressure sensor) may be located on the inner side of the flexible strap 300 to measure force exerted by the chest/abdomen 700 as the subject inhales and exhales. Because the flexible strap 300 does not lengthen, the flexible strap 300 will not attenuate the force exerted by the chest/abdomen 700 on the sensor 310.

The guide assembly 400 guides motion of the reel assembly 500 toward and away from the backrest 104. In the example embodiment shown, the guide assembly includes multiple (e.g., four) parallel guide rods 402 that are secured to the front plate 104 and the rear plate 106 and that hold the front plate 104 and the rear plate 106 in position. The reel assembly 500 may be secured to linear bearings 404 that are free to translate along the guide rods 402. In an embodiment, guide rods 402 are eighteen (18) inches long and the guide rods 402 on one side are separated from guide rods 402 on the other side by twenty-four (24) inches. However, other guide configurations known to the artisan may be used.

The reel assembly 500 includes a first attachment assembly 502 and a second attachment assembly 504, each configured to connect to the flexible strap 300 on opposite sides of the loop 306. The first attachment assembly 502 and the second attachment assembly 504 are secured to a reel assembly plate 506.

The second attachment assembly 504 may secure to the flexible strap 300 at one place on the flexible strap 300 and not permit relative movement between the second attachment assembly 504 and the flexible strap 300.

The first attachment assembly 502 may be configured as a reel assembly 510 that is configured to function as a reel for the flexible strap 300. The reel assembly 510 includes a spindle 512 around which the flexible strap 300 can be wound via a handle 514 to tighten the flexible strap 300 around the chest/abdomen 700. In an example embodiment, the reel assembly 510 is a ratcheting reel assembly and includes a ratchet gear 516 and a pawl 518 that operate in the conventional ratcheting manner. As the operator turns the handle 514, the flexible strap 300 wraps around the spindle 512. As the flexible strap 300 is wound around the spindle 512, the pawl 518 cooperates with the ratchet gear 516 to prevent the flexible strap 300 from unwinding from the spindle 512. Once the desired tension is applied to the flexible strap 300, the ratchet gear 516 and pawl 518 hold the flexible strap 300 in place and thereby maintain the desired tension in the flexible strap 300. To release the tension, the operator simply needs to manually disengage the pawl 518 from the ratchet gear 516.

The reel assembly 500 further includes a width adjustment assembly 520 configured to enable adjustment of a width W between the first attachment assembly 502 and the second attachment assembly 504. The width adjustment assembly 520 includes parallel guide rails 522 secured to the reel assembly plate 506 and respective carriages 524 secured to the first attachment assembly 502 and the second attachment assembly 504. The carriages 524 enable the first attachment assembly 502 and the second attachment assembly 504 to translate unrestricted along the guide rails 522 and thereby change the width W therebetween. Once a suitable width W is reached, the associated positions of the carriages 524 may (optionally) be fixed relative to the guide rails 522 via, for example, carriage set screws 524S. One or more backrest slots 104S (e.g., 0.5″ wide and positioned every inch) permit the flexible strap 300 to pass through the backrest 104 regardless of the width W between the first attachment assembly 502 and the second attachment assembly 504.

The anchoring assembly 600 provides an anchoring function for the reel assembly 500 which is otherwise free to translate along the guide rods 402 unrestricted. The anchoring assembly 600 is configured to provide a static anchoring function, to provide a dynamic anchoring function, or to provide both a static anchoring function and a dynamic anchoring function, one at a time.

To provide the static anchoring of the anchoring assembly 500, the anchoring assembly 600 includes a static load assembly 610 that locks the reel assembly 500 into a fixed position relative to/along the guide rods 402. Locking the reel assembly 500 into the fixed position relative to the guide rods 402 also locks the reel assembly 500 into a fixed position relative to the backrest 104 and likewise into a fixed position relative to the chest/abdomen 700 of the subject resting against the backrest 104.

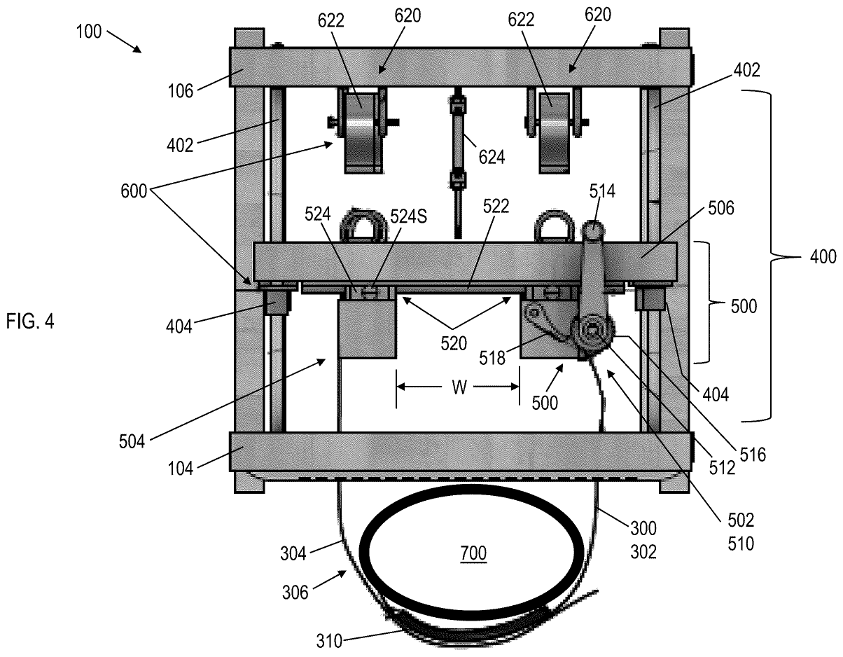

When the reel assembly 500 is locked into this fixed position, first attachment assembly 502 and the second attachment assembly 504 of the reel assembly 500 are likewise locked into the fixed position as is the flexible strap 300 that is attached thereto. In this fixed position, when the flexible strap 300 is tightened around a subject, attempts by the subject to expand the chest/abdomen 700 are resisted by the flexible strap 300 which does not move nor stretch. When the anchoring assembly 500 is providing static anchoring, tension in the flexible strap 300 increases as effort to expand the chest/abdomen 700 increases and decreases as the chest/abdomen 700 contracts. FIG. 4, when the reel assembly 500 fixed into the position as shown, represents a static anchoring configuration where the flexible strap 300 is subject to the static anchoring. Information provided by the sensor 310 during static anchoring can then be gathered to determine the effects on the chest/abdomen 700.

The static load assembly 610 may include at least one locking linear bearing 404L among the linear bearings 404. The locking linear bearing 404L may include, for example a set screw that fixes the locking linear bearing 404L to the respective guide rod 402.

To provide the dynamic anchoring of the anchoring assembly 600, the anchoring assembly 600 includes a dynamic load assembly 620 that moves with the chest/abdomen 700 as the chest/abdomen 700 expands and contracts and thereby provides a constant load on the chest/abdomen 700, regardless of where the chest/abdomen 700 is in an expansion/contraction cycle.

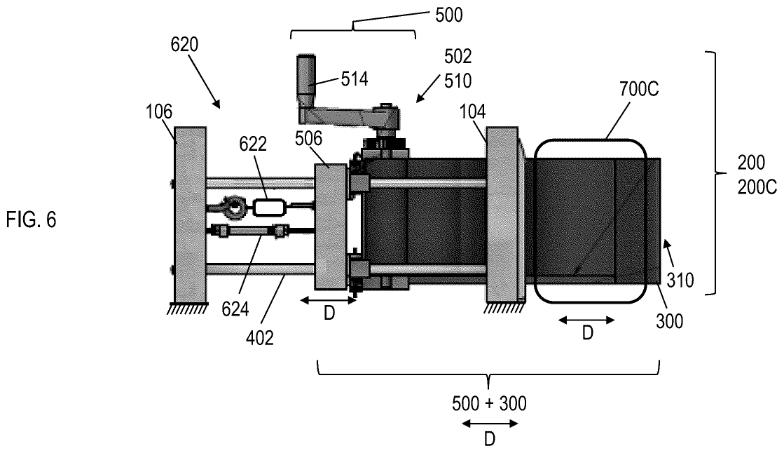

FIG. 6 shows the chest restriction assembly 200C of the restriction device 100 of FIG. 1 and is also representative of an example embodiment of the abdomen restriction assembly 200A.

As can be seen in FIG. 6, the dynamic load assembly 620 includes a constant load element 622 (e.g., a constant force spring) and a displacement sensor 624 (e.g., a linear variable differential transducer sensor (LVDT)). In an example embodiment, the constant load element 622 comprises a flat strip of spring material formed into a coil. The spring may reach its full strength after being stretched to 1.25 times its diameter and thereafter exert a relatively steady force no matter how much farther it is stretched. A spring constant of the spring may be selected as desired. The displacement sensor 624 measures the horizontal displacement of the chest 700C during breathing when a constant load is applied by the constant load element 622.

To apply the constant load, a constant load element 622 is selected that provides the desired load. The constant load element 622 is connected between the rear plate 106 and the reel assembly plate 506. The flexible strap 300 is tightened around the chest 700C. (The locking linear bearings 404L are not locked into a fixed position along the guide rod 402.)

Once connected to the reel assembly plate 506, the constant load element 622 exerts a constant load that pulls the reel assembly 500 toward the rear plate 106. The reel assembly 500 is able to translate on the guide rods 402 and the flexible strap 300 moves with the reel assembly 500. When the flexible strap 300 moves with the reel assembly 500, the flexible strap 300 transfers the constant load to the chest 700C. The reel assembly 500 and the flexible strap 300 remain free to displace along the guide rods 402 while the constant load is being applied to the reel assembly 500.

The constant load that is selected will be sufficient to mitigate but will not be sufficient to prevent horizontal movement (displacement) of the chest 700C, which is represented by the double arrow D. Consequently, the reel assembly 500 and the flexible strap 300 will displace horizontally with the chest 700C as the chest 700C expands and contracts throughout the entire range of the displacement D.

The constant load element 622 exerts a constant load on the reel assembly 500 throughout a wide range of the position of the reel assembly 500 along the guide rods 402. The reel assembly 500 and its associated displacements D can thereby be positioned within the operating range of the displacement sensor 624 simply by adjusting the length of the flexible strap 300. When the reel assembly 500 is positioned within the operating range of the displacement sensor 624, the displacement D of the reel assembly 500 can be measured. Since the displacement D of the reel assembly 500 is the same as the displacement of the flexible strap 300, and since the displacement of the flexible strap 300 is the same as the displacement of the chest 700C, the displacement sensor 624 can measure the displacement of the chest 700C during expansion and contraction. The information provided by the displacement sensor 624, together with the information provided by the sensor 310, can then be gathered to determine the effects of the selected constant load on the chest 700C.

In an example embodiment, a first step in setting up a subject involves measuring the subject's height and torso width. The subject then sits on an adjustable seat, initially measuring from the floor to the seat sixteen (16) inches and extending to a total height of twenty-three (23) inches. The bench is adjusted based on the subject's chest and abdomen. The subject sits with their back against the front of the restriction device 100, pressing onto the front plate 104 or the cushioning 104C, and the flexible straps 300 are adjusted around the chest and abdomen respectively to achieve a customized circumferential fit, to include passing the flexible straps 300 through appropriate slots 104S. A goal is to align the flexible straps 300 parallel to the subject's body width, with adjustments made possible by moving first attachment assembly 502 and the second attachment assembly 504.

Once the subject is fully strapped into and positioned on the device, tension is created using the respective reel assemblies 500. Adjusting the reel assemblies 500 to different levels applies varying pressure to the subject's abdomen and chest, simulating dynamic chest wall restriction. In contrast, the constant load elements 622 are disconnected for a static simulation, which allows the two reel assembly plates 506 to be pulled back to the desired position while being restricted by the displacement sensor 624. Then the locking linear bearings 404L are used to secure the reel assembly plates 506 in place to create the static simulation.

The restriction device 100 was used in two separate experiments to evaluate its performance. A goal of the first experiment (dynamic anchoring-constant force) was to mimic hydrostatic pressure that divers face when underwater. Seven males and two females (mean age 26.5±6.1 years), all in good health, participated.

The study protocol consisted of two cognitive tasks administered under three distinct conditions: baseline (without load), a load of thirty (30) kg, and a load of forty-five (45) kg. The subjects were assigned to perform the tasks, with the baseline condition consistently occurring first, followed by the conditions with loads. The order of the load conditions and the order of the cognitive tasks were randomized to mitigate potential order effects across subjects. The loaded conditions also included, in series, a twenty-five (25) cmH20/L/s linear resistance and a pressure threshold valve set at twenty percent (20%) of the subjects'Maximum Inspiratory Pressure (MIP), all connected to the inspiratory side of a breathing valve.

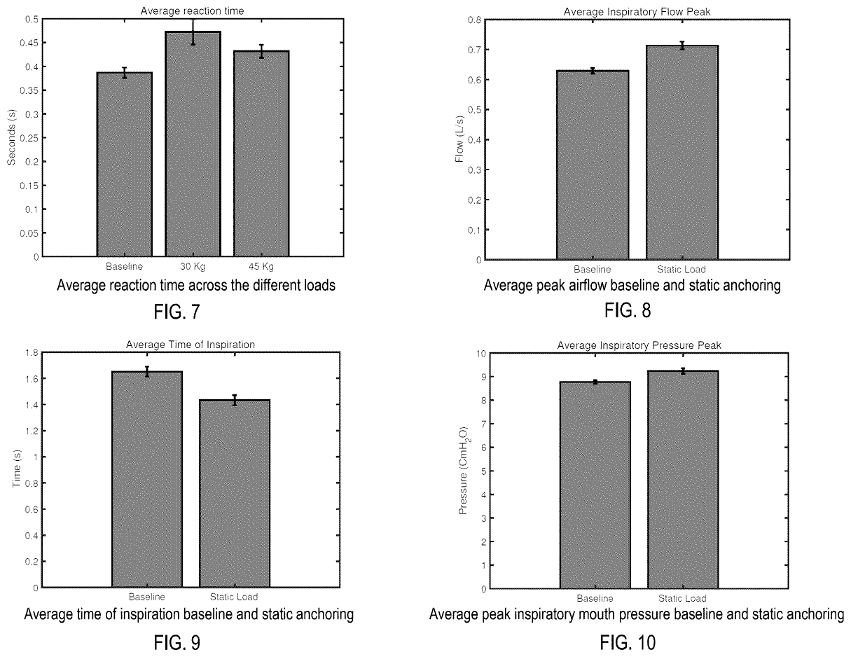

The average reaction time of subjects across the three different loads using a Psychomotor Vigilance Test were compared. FIG. 7 shows the average reaction time across the different loads. The Kruskal-Wallis test revealed statistically significant differences between the groups, with a p 0.001< for baseline vs. thirty (30) kg and baseline vs. forty-five (45) kg. However, no significant difference was found between thirty (30) kg and forty-five (45) kg, with a p-value of 0.81661.

In a second experiment, static anchoring was used, and only the chest was restricted. Airflow and mouth pressure during normal breathing were recorded both during baseline conditions and under the static anchoring. Statistical analysis revealed significant differences in the average peak airflow, peak pressure, and time of inspiration between baseline and under load conditions (p0.001). FIG. 8 compares the average peak<airflow between baseline and static anchoring conditions, showing an increase in the peak airflow. FIG. 9 shows a decrease in the average time of inspiration under static anchoring conditions versus baseline. FIG. 10 reveals the average peak inspiratory mouth pressure increase between baseline and static anchoring conditions. The peak airflow and pressure increase and Ti decrease suggest an adaptive response to compensate for the added respiratory impedance. It is hypothesized that the need to overcome the chest restriction required the subjects to engage in deeper and more forceful breathing patterns, resulting in a quicker and more forceful inspiratory breath than baseline.

The restriction device 100 offers a novel design to study the impact of chest wall impairment on breathing and cognition. It is an improvement on earlier devices, offering improved precision and versatility while allowing for accurate quantification of the load applied to the chest wall and abdomen. Thus, it provides researchers with controlled conditions for exploring the impacts of static and dynamic load-based restrictions on physiological and cognitive processes and modeling breathing dynamics under load [5]. These validation studies have demonstrated significant experimental manipulation in both physiological and cognitive functions. Additional use cases for the restriction device 100 include respiratory training, assisting patients with respiratory conditions such as neuromuscular diseases [6], [7], and reducing the effort required to breath [8], [9].

The present Inventors have created a unique and innovative restriction device that allows for setting of known loads independently to the chest and abdomen, during which individual force and displacement data can be gathered. The restriction device thereby represents an improvement in the art.

All features disclosed in the specification, including the claims, abstract, and drawings, and all the steps in any method or process disclosed, may be combined in any combination, except combinations where at least some of such features and/or steps are mutually exclusive. Each feature disclosed in the specification, including the claims, abstract, and drawings, can be replaced by alternative features serving the same, equivalent, or similar purpose, unless expressly stated otherwise.

While various embodiments of the present invention have been shown and described herein, it will be obvious that such embodiments are provided by way of example only. Numerous variations, changes and substitutions may be made without departing from the invention herein. Accordingly, it is intended that the invention be limited only by the spirit and scope of the appended claims.

[1] F. Zechman and R. Wiley, “Effect of chest cage restriction on perception of added airflow resistance,” Respiration Physiology, vol. 31, pp. 71-79, September 1977.

[2] M. Eberlein, G. A. Schmidt, and R. G. Brower, “Chest wall strapping. An old physiology experiment with new relevance to small airways diseases,” Annals of the American Thoracic Society, vol. 11, pp. 1258-1266, October 2014.

[3] G. W. Sybrecht, L. Garrett, and N. R. Anthonisen, “Effect of chest strapping on regional lung function,” Journal of Applied Physiology, vol. 39, pp. 707-713, November 1975.

[4] C. Cline, J. Coast, and D. Arnall, “A chest wall restrictor to study effects on pulmonary function and exercise,” Respiration, vol. 66, no. 2, p. 182-187, 1999.

[5] N. J. Napoli, V. R. Rodrigues, and P. W. Davenport, “Characterizing and Modeling Breathing Dynamics: Flow Rate, Rhythm, Period, and Frequency,” Frontiers in Physiology, vol. 12, 2022.

[6] K. Watson, T. Egerton, N. Sheers, S. Retica, R. McGaw, T. Clohessy, P. Webster, and D. J. Berlowitz, “Respiratory muscle training in neuromuscular disease: a systematic review and meta-analysis,” European Respiratory Review, vol. 31, p. 220065, November 2022.

[7] V. R. Rodrigues, W. L. Olsen, E. Sajjadi, B. K. Smith, and N. J. Napoli, “Exploring inspiratory occlusion metrics to assess respiratory drive in patients under acute intermittent hypoxia,” Respiratory Physiology & Neurobiology, vol. 304, p. 103922, October 3022.

[8] K. Neumannova, V. Kuzilkova, M. Zurkov'a, L. Hubackova, T. Michalcikova, P. Jakubec, and V. Kolek, “Respiratory muscle training improves the work of breathing and decreases inspiratory muscle fatigue in patients after lung transplantation,” in Physiotherapists, European Respiratory Society, September 2019.

[9] V. Ribeiro Rodrigues, S. L. Beres, P. W. Davenport, and N. J. Napoli, “Modeling and analysis of mechanical work of breathing,” in Handbook of AI and Data Sciences for Sleep Disorders (R. B. Berry, P. M. Pardalos, and X. Xian, eds.), Springer, 2024.

1. An apparatus, comprising:
a backrest; and
at least one restriction assembly comprising: a flexible strap configured to wrap around a subject abutting a front of the backrest; a reel assembly disposed behind the backrest and configured to tighten the flexible strap wrapped around the subject; and an anchor assembly configured to hold the reel assembly apart from the backrest.

2. The apparatus of claim 1,
wherein the backrest comprises at least one opening configured to permit the flexible strap to pass therethrough.

3. The apparatus of claim 1,
wherein the flexible strap comprises a pressure sensor.

4. The apparatus of claim 1,
wherein the reel assembly comprises a ratcheting reel assembly.

5. The apparatus of claim 1,
wherein the reel assembly is configured to permit adjustment of a width of the flexible strap to conform to a width of the subject.

6. The apparatus of claim 1,
wherein the anchor assembly comprises a static load assembly configured to fix the reel assembly into a fixed position relative to the backrest.

7. The apparatus of claim 6,
further comprising a guide assembly configured to enable movement of the reel assembly toward and away from the backrest;
wherein the static load assembly is configured to lock the reel assembly into the fixed position along the guide assembly.

8. The apparatus of claim 1,
wherein the anchor assembly comprises a dynamic load assembly configured to apply a constant load to the reel assembly in a direction away from the backrest to hold the reel assembly apart from the backrest.

9. The apparatus of claim 8,
wherein the dynamic load assembly comprises a constant force spring configured to apply the constant load.

10. The apparatus of claim 1,
further comprising a displacement sensor configured to measure displacement of the reel assembly.

11. The apparatus of claim 1, wherein the at least one restriction assembly comprises:
a chest restriction assembly configured to wrap around a chest of the subject; and
an abdomen restriction assembly configured to wrap around an abdomen of the subject;
wherein the chest restriction assembly and the abdomen restriction assembly are configured to operate independently of each other.

12. An apparatus, comprising:
a backrest; and
at least one restriction assembly comprising: a flexible strap configured to form a closed loop around a subject abutting a front of the backrest; a guide assembly disposed behind the backrest; a reel assembly disposed on the guide assembly, configured to move toward and away from the backrest along the guide assembly, and configured to operate as a reel for the flexible strap; and an anchor assembly configured to hold the reel assembly apart from the backrest.

13. The apparatus of claim 12,
wherein the flexible strap comprises a pressure sensor.

14. The apparatus of claim 12, wherein the reel assembly comprises:
a first attachment assembly configured to connect to the flexible strap; and
wherein the first attachment assembly comprises a ratchet assembly configured to reel the flexible strap in to apply tension to the flexible strap.

15. The apparatus of claim 12, wherein the reel assembly comprises:
a first attachment assembly configured to connect to the flexible strap;
a second attachment assembly configured to connect to the flexible strap; and
a width adjustment assembly configured to enable adjustment of a width between the first attachment assembly and the second attachment assembly.

16. The apparatus of claim 15,
wherein the width adjustment assembly comprises a width adjustment rail; and
wherein the first attachment assembly and the second attachment assembly are slidably connected to the width adjustment rail in a manner effective to permit the adjustment of a distance therebetween to adjust the width.

17. The apparatus of claim 12,
wherein the anchor assembly comprises a static load assembly configured to fix the reel assembly into a fixed position relative to the guide assembly and thereby relative to the backrest.

18. The apparatus of claim 12,
wherein the anchor assembly comprises a dynamic load assembly configured to apply a constant load to the reel assembly in a direction away from the backrest and along the guide assembly to hold the reel assembly apart from the backrest.

19. The apparatus of claim 12,
further comprising a displacement sensor configured to measure displacement of the reel assembly.

20. The apparatus of claim 12, wherein the at least one restriction assembly comprises:
a chest restriction assembly configured to wrap around a chest of the subject; and
an abdomen restriction assembly configured to wrap around an abdomen of the subject;
wherein the chest restriction assembly and the abdomen restriction assembly are configured to operate independently of each other.
